(19) United States
(12) Patent Application Publication
(10) Pub. No.: US 2021/0208071 A1
(43) Pub. Date: Jul. 8, 2021

(54) METHODS FOR WATERMARKING AND IDENTIFYING CHEMICAL COMPOSITIONS

(71) Applicant: ChromaCode, Inc., Carlsbad, CA (US)

(72) Inventors: Aditya RAJAGOPAL, Carlsbad, CA (US); Alexander DICKINSON, Carlsbad, CA (US); Forest BASKETT, Carlsbad, CA (US)

(21) Appl. No.: 17/058,550

(22) PCT Filed: Jun. 3, 2019
(86) PCT No.: PCT/US2019/035129

§ 371 Date: Nov. 24, 2020

Related U.S. Application Data
(60) Provisional application No. 62/680,426, filed on Jun. 4, 2018.

(51) Int. Cl. G01N 21/64; C12Q 1/6876; G01N 21/76

(52) U.S. Cl. CPC G01N 21/6428; C12Q 1/6876; G01N 2021/6441; C12Q 2600/16; G01N 21/76

(57) ABSTRACT

The present disclosure provides methods and compositions to “watermark” chemical compounds. The disclosed methods provide methods whereby the unique watermark allows identification of substantially similar chemical reactions in a single qPCR reaction mixture. Further, the disclosure provides methods whereby the unique watermark allows for identification of reaction mixtures.

### CROSS-REFERENCE

This application claims the benefit and priority to U.S. Provisional Patent Application No. 62/680,426, filed Jun. 4, 2018, and is incorporated by reference in their entirety for all purposes.

### BACKGROUND

Watermarking is a method used to identify original works fixed in a tangible medium by encoding identifiers, distinct from the information content itself, into the work. These identifiers are used to authenticate the originality of the work or objects themselves.

### SUMMARY

Disclosed herein, in some aspects, is a method of verifying the synthesis of a chemical mixture, said method comprising: (a) providing a sample comprising: (i) a chemical mixture, comprising a first chemical component and a second chemical component; (ii) a first watermarking agent configured to emit a first detectable signal, where said first watermarking agent indicates the presence of said first chemical component; and (iii) a second watermarking agent configured to emit a second detectable signal, where said second watermarking agent indicates the presence of said second chemical component; and (b) detecting the presence of said first detectable signal and said second detectable signal, thereby verifying the synthesis of said chemical composition. In some embodiments, the method comprises obtaining a time stamp. In some embodiments, the time stamp indicates a time of synthesis. In some embodiments, the method comprises outputting a report with a value of said first detectable signal, a value of said detectable signal and said time stamp. In some embodiments, the method comprises obtaining a geo-tag. In some embodiments, the geo-tag indicates a location of verification of synthesis or a location of synthesis.

Disclosed herein, in some aspects, is a method of tracking a batch of chemical mixture, said method comprising: (a) providing chemical mixture comprising: (i) a chemical mixture; and (ii) a first watermarking agent configured to emit a first detectable signal; (b) detecting said first detectable signal; and (c) comparing said first detectable signal to a reference signal indicative of a batch of a chemical mixture. In some embodiments, the method comprises a second watermarking agent configured to emit a second detectable signal. In some embodiments, the method comprises calculating a ratio of said first detectable signal and said second detectable signal. In some embodiments, the method comprises comparing said ratio to a reference ratio indicative of a batch of a chemical mixture. In some embodiments, the method comprises obtaining a geo-tag. In some embodiments, the method comprises outputting a report indicative of the presence of said batch at a location indicated by said geo-tag. In some embodiments, the method comprises obtaining a time stamp. In some embodiments, the reference signal is indicative of a time of manufacture of said batch. In some embodiments, the method comprises outputting a report. In some embodiments, the report indicates the presence of a batch at a time of said time stamp. In some embodiments, the report indicates of an age of said batch.

Disclosed herein, in some aspects, is a method of watermarking a chemical composition, the method comprising: (a) obtaining a chemical composition; and (b) introducing into said chemical composition a watermarking agent that is configured to produce a detectable signal upon application of reaction conditions to the chemical composition, the presence of said detectable signal indicating that the chemical composition has been watermarked. In some embodiments, the detectable signal is dependent on a concentration of said watermarking agent. In some embodiments, the detectable signal is configured to be compared with or compared to a reference signal. In some embodiments, the watermarking agent is a selected from the group consisting of: (a) a dye, (b) a fluorescent molecule, (c) a chemiluminescent label, (d) a magnetic particle, (e) an electret structure exhibiting a permanent dipole, (f) a radioactive species, and any combination thereof. In some embodiments, the watermarking agent absorbs electromagnetic radiation. In some embodiments, the watermarking agent emits electromagnetic radiation. In some embodiments, the watermarking agent comprises a quenching agent. In some embodiments, the watermarking agent comprises a metal. In some embodiments, the watermarking agent comprises a nucleic acid. In some embodiments, the watermarking agent comprises an enzyme. In some embodiments, the watermarking agent is an enzyme substrate. In some embodiments, the watermarking agent is an enzyme substrate configured to be processed to produce said detectable signal. In some embodiments, the watermarking agent comprises one or more components for performing a reaction that produces said detectable signal. In some embodiments, the components comprise nucleic acids and said reaction comprises a nucleic acid extension. In some embodiments, the watermarking agent configured to produce said detectable signal upon solvation of said watermarking agent. In some embodiments, the detectable signal comprises electromagnetic radiation. In some embodiments, the electromagnetic radiation comprises light. In some embodiments, the detectable signal comprises electromagnetic radiation at a particular wavelength. In some embodiments, the detectable signal comprises electromagnetic radiation at a plurality of wavelengths. In some embodiments, the detectable signal comprises a magnetic field. In some embodiments, the detectable signal comprises an electric field. In some embodiments, the detectable signal comprises a radioactive decay. In some embodiments, the detectable signal comprises a change in signal from a baseline signal. In some embodiments, the change in signal from a baseline signal comprises a decrease in signal amplitude. In some embodiments, the method further comprises introducing a second watermarking agent. In some embodiments, the combination of the first and second watermarking agents generates said detectable signal upon application of said reaction conditions to the chemical composition. In some embodiments, the second watermarking agent is configured to produce a second detectable signal upon application of reaction conditions to the chemical composition. In some embodiments, the chemical composition comprises said watermarking agent at a first concentration and said second watermarking agent at a second concentration. In some embodiments, the watermarking agent and said second watermarking reagent are configured to produce a detectable signal and a second detectable signal at a ratio of said detectable signal and a second detectable signal; where said ratio indicates that the chemical composition has been watermarked. In some embodiments, the watermarking agent and said second watermarking agent are configured to be detected simultaneously. In some embodiments, the watermarking agent and said second watermarking agent are configured to be detected sequentially. In some embodiments, the watermarking agent and said second watermarking agent are configured to be detected using the same instrument. In some embodiments, the watermarking agent and said second watermarking agent are configured to be detected using the same fluorescent channel. In some embodiments, the chemical composition further comprises assay reagent(s). In some embodiments, the assay reagent(s) comprise reagent(s) for a nucleic acid extension reaction. In some embodiments, the nucleic acid extension reaction is polymerase chain reaction (PCR). In some embodiments, the assay reagent(s) comprise a buffer, a salt, or an enzyme. In some embodiments, the polymerase chain reaction is a quantitative polymerase chain reaction (qPCR). In some embodiments, the assay reagent(s) comprises a DNA polymerase, a reverse transcriptase, an RNA polymerase, or a combination thereof. In some embodiments, the assay reagent(s) comprise a dNTP, a salt, a buffer, or a combination thereof. In some embodiments, the assay reagents(s) comprise an oligonucleotide primer. In some embodiments, the oligonucleotide primer is configured to target a nucleic acid sequence. In some embodiments, the oligonucleotide primer is configured to hybridize to a nucleic acid sequence. In some embodiments, the assay reagent(s) comprise an oligonucleotide probe. In some embodiments, the oligonucleotide probe is a TaqMan® probe. In some embodiments, the oligonucleotide probe is a molecular beacon. In some embodiments, the oligonucleotide probe comprises a label. In some embodiments, the label and said watermarking agent comprise the same molecular structure. In some embodiments, the label and said watermarking agent comprise a different molecular structure. In some embodiments, the label produces said detectable signal.

Disclosed herein, in some aspects, is a method of identifying a chemical composition, said method comprising: (a) obtaining a chemical composition comprising: (i) a first watermarking agent configured to emit a first detectable signal; and (ii) a second watermarking agent configured to emit a second detectable signal; (b) detecting said first detectable signal and said second detectable signal; (c) calculating a ratio of said first detectable signal and said second detectable signal; and (d) comparing said ratio to a reference ratio indicative of said chemical composition of chemical reagents, thereby identifying said chemical composition. In some embodiments, the ratio is dependent on a concentration of said first watermarking agent and a concentration of said second watermarking agent. In some embodiments, the chemical composition further comprises nucleic acids. In some embodiments, the method comprises performing a nucleic acid extension reaction. In some embodiments, the extension reaction is a polymerase chain reaction. In some embodiments, the polymerase chain reaction is a quantitative polymerase chain reaction. In some embodiments, the chemical composition comprises a mixture of polymerase chain reaction reagents comprising: a buffer, a salt, a dNTP, an enzyme, or a combination thereof. In some embodiments, the salt comprises magnesium, sodium, potassium, chloride, or citrate. In some embodiments, the enzyme is selected from the group consisting of DNA polymerase, RNA polymerase, reverse transcriptase. In some embodiments, the nucleic acids comprise a first set of oligomers configured to amplify a first sequence and a first probe comprising said first watermarking agent configured to emit said first detectable signal upon amplification of said first sequence. In some embodiments, the first probe is a nucleic acid. In some embodiments, the first probe hybridizes to at least a portion of said first sequence. In some embodiments, the first detectable watermarking agent provides a quantitative ratio measurement corresponding to an abundance of said a first target sequence in the sample. In some embodiments, the nucleic acids comprise a second set of oligomers configured to amplify a second sequence and a second probe comprising said second watermarking agent configured to emit said second detectable signal upon amplification of said second sequence. In some embodiments, the second probe is a nucleic acid. In some embodiments, the second probe hybridizes to at least a portion of said second sequence. In some embodiments, the second detectable watermarking agent provides a quantitative ratio measurement corresponding to an abundance of a second target sequence in the sample. In some embodiments, the first probe is a TaqMan® probe. In some embodiments, the first probe is a molecular beacon or molecular torch. In some embodiments, the first probe and said second probe is a TaqMan® probe. In some embodiments, the first probe and said second probe is a molecular beacon or molecular torch. In some embodiments, the method comprises subsequent to detecting said first detectable signal and said second detectable signal, detecting at a second time point said first detectable signal and said second detectable signal. In some embodiments, the method comprises subsequent to said second time point, processing said first detectable signal to determine a presence or concentration of said first sequence. In some embodiments, the method comprises subsequent to said second time point, processing said second detectable signal to determine a presence or concentration of said second sequence. In some embodiments, the chemical composition comprises a chemical reagent. In some embodiments, the chemical composition comprises two chemical reagents. In some embodiments, the reaction conditions are polymerase chain reaction (PCR) conditions.

Disclosed herein, in some aspects, is a watermarked chemical composition, comprising: (a) one or more chemical components; and (b) a watermarking agent that is configured to produce a detectable signal upon application of reaction conditions to the composition, the presence of said detectable signal indicating that the composition has been watermarked. In some embodiments, the detectable signal is dependent on a concentration of said watermarking agent. In some embodiments, the detectable signal is configured to be compared with or compared to a reference signal. In some embodiments, the watermarking agent is a selected from the group consisting of: (a) a dye, (b) a fluorescent molecule, (c) a chemiluminescent label, (d) a magnetic particle, (e) an electret structure exhibiting a permanent dipole, (f) a radioactive species, and any combination thereof. In some embodiments, the watermarking agent absorbs electromagnetic radiation. In some embodiments, the watermarking agent emits electromagnetic radiation. In some embodiments, the watermarking agent comprises a quenching agent. In some embodiments, the watermarking agent comprises a metal. In some embodiments, the watermarking agent comprises a nucleic acid. In some embodiments, the watermarking agent comprises an enzyme. In some embodiments, the watermarking agent is an enzyme substrate. In some embodiments, the watermarking agent is an enzyme substrate configured to be processed to produce said detectable signal. In some embodiments, the watermarking agent comprises one or more components for performing a reaction that produces said detectable signal. In some embodiments, the components comprise nucleic acids and said reaction comprises a nucleic acid extension. In some embodiments, the watermarking agent is configured to produce said detectable signal upon solvation of said watermarking agent. In some embodiments, the detectable signal comprises electromagnetic radiation. In some embodiments, the electromagnetic radiation comprises light. In some embodiments, the detectable signal comprises electromagnetic radiation at a particular wavelength. In some embodiments, the detectable signal comprises electromagnetic radiation at a plurality of wavelengths. In some embodiments, the detectable signal comprises a magnetic field. In some embodiments, the detectable signal comprises an electric field. In some embodiments, the detectable signal comprises a radioactive decay. In some embodiments, the detectable signal comprises a change in signal from a baseline signal. In some embodiments, the change in signal from a baseline signal comprises a decrease in signal amplitude. In some embodiments, the watermarked chemical composition comprises introducing a second watermarking agent. In some embodiments, the combination of said first and second watermarking agents generates said detectable signal upon application of said reaction conditions to the chemical composition. In some embodiments, the second watermarking agent is configured to produce a second detectable signal upon application of reaction conditions to the chemical composition. In some embodiments, the chemical composition comprises said watermarking agent at a first concentration and said second watermarking agent at a second concentration. In some embodiments, the watermarking agent and said second watermarking reagent are configured to produce a detectable signal and a second detectable signal at a ratio of said detectable signal and a second detectable signal; where said ratio indicates that the chemical composition has been watermarked. In some embodiments, the watermarking agent and said second watermarking agent are configured to be detected simultaneously. In some embodiments, the watermarking agent and said second watermarking agent are configured to be detected sequentially. In some embodiments, the watermarking agent and said second watermarking agent are configured to be detected using the same instrument. In some embodiments, the watermarking agent and said second watermarking agent are configured to be detected using the same fluorescent channel. In some embodiments, the chemical composition further comprises assay reagent(s). In some embodiments, the assay reagent(s) comprise reagent(s) for a nucleic acid extension reaction. In some embodiments, the nucleic acid extension reaction is polymerase chain reaction (PCR). In some embodiments, the assay reagent(s) comprise a buffer, a salt, or an enzyme. In some embodiments, the polymerase chain reaction is a quantitative polymerase chain reaction (qPCR). In some embodiments, the assay reagent(s) comprises a DNA polymerase, a reverse transcriptase, an RNA polymerase, or a combination thereof. In some embodiments, the assay reagent(s) comprise a dNTP, a salt, a buffer, or a combination thereof. In some embodiments, the assay reagents(s) comprise an oligonucleotide primer. In some embodiments, the oligonucleotide primer is configured to target a nucleic acid sequence. In some embodiments, the oligonucleotide primer is configured to hybridize to a nucleic acid sequence. In some embodiments, the assay reagent(s) comprise an oligonucleotide probe. In some embodiments, the oligonucleotide probe is a TaqMan® probe. In some embodiments, the oligonucleotide probe is a molecular beacon. In some embodiments, the oligonucleotide probe comprises a label. In some embodiments, the label and said watermarking agent comprise the same molecular structure. In some embodiments, the label and said watermarking agent comprise a different molecular structure. In some embodiments, the label produces said detectable signal.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example watermarking in a two-channel qPCR experiment.

FIGS. 2A-2C illustrates an example watermarking in a four-color qPCR experiment.

FIG. 3 illustrates an example workflow for validating a qPCR assay.

### DETAILED DESCRIPTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

Polymerase Chain Reaction (PCR) is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2 fold with each cycle leading to approximately a billion-fold increase of the target sequence in 30 cycles (Saiki et al 1988).

Primers, or “amplification oligomers,” used herein interchangeably, refer to an oligonucleotide or nucleic acid configured to bind to another nucleic acid and facilitate one or more reactions, for example, transcription, nucleic acid synthesis, and nucleic acid amplification. A primer can be double-stranded. A primer can be single-stranded. A primer can be a forward primer or a reverse primer. A forward primer and a reverse primer can be those which bind to opposite strands of a double-stranded nucleic acid. For example, a forward primer can bind to a region of a first strand (e.g., Watson strand) derived from a nucleic acid, and a reverse primer can bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid, or may bind to the non-coding strand of a nucleic acid. A reverse primer may bind to the coding strand of a nucleic acid, or may bind to the non-coding strand of a nucleic acid.

Frequently, the target-specific oligonucleotide probe is a short oligonucleotide complementary to one strand of the amplified target. The probe lacks a 3′ hydroxyl and therefore is not extendable by the DNA polymerase. TaqMan® (ThermoFisher Scientific) chemistry is a common reporter probe method used for multiplex Real-Time PCR (Holland et al. 1991). The TaqMan oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

### Overview

Disclosed herein are methods, composition, and systems used for watermarking. Watermarking may allow the creation of a “chemical fingerprint” such that the composition has an identifier that can be chosen for a chemical composition and does not rely on the observation of the chemical composition itself. Watermarking of chemical compositions may be advantageous in the ability to track and identify chemical compositions the may be otherwise difficult to do. Some chemical compositions may comprise a mixture of mixture components and the identity and concentration of the mixture components may difficult to quickly and easily ascertain. Even with the use of spectroscopic tools to identify the identity of a mixture component, for example, nuclear magnetic resonance spectroscopy (NMR), infrared radiation spectroscopy (IR), Raman spectroscopy, mass spectrometry (MS), it may be time consuming and require purification of the components prior to performing spectroscopy. Moreover, the quantification of the concentration of the mixture component may be difficult to perform, especially in differentiating between similar concentrations. In the case of many molecular biology reagents, the concentrations of the similar mixtures may differ on the order of micromolar, requiring the quantitative tools to have sufficient resolution in order to differentiate the chemical compositions from one another. Watermarking the chemical composition may allow a rapid and facile test to be performed in which a watermark may be identified. Once the watermarked has been identified, characteristics of the chemical composition that have been associated with a specific watermark can be ascertained.

Disclosed herein, is a method of watermarking a chemical composition. The method may comprise introducing a watermarking agent to watermark the chemical composition. The watermarking agent may be configured to emit a detectable signal. The watermarking agent may be configured to emit a detectable signal when subjected to reaction conditions. Detection of the detectable signal may be used to identify that the chemical composition has been watermarked.

Disclosed herein, is method of using distinct ratios of watermarking agents to watermark chemical compounds. The individual watermarking agents may emit a signal at an amplitude and the amplitude the signals may be determined. The ratio of the signal amplitudes may be used as the “watermark”, such that the calculated ratio may be used to identify or track a chemical composition, or be otherwise be used in methods as disclosed herein as a watermarking agent.

Disclosed herein, is a method of using watermarking to track the synthesis of a chemical mixture. The method may comprise indexing a plurality of watermarking agents with a plurality of reagents. The plurality of watermarking agents may be indexed such that a watermarking agent is paired with a reagent. The plurality of watermarking agents may be indexed such that a watermarking agent is paired with only one reagent. The method may comprise pairing a first watermarking agent with first reagent. The first watermarking may be present only when the first reagent is in the chemical mixture. The method may further comprise pairing a second watermarking agent with a second reagent. The method may further comprise detecting the first watermarking reagent, thereby determining that the first reagent is present in the chemical mixture. The method may further comprise detecting a second watermarking reagent, thereby detecting that the second reagent is present in the chemical mixture. The signal amplitudes of the watermarking agents may also be used such that the amplitudes of the watermarking agents correspond to the concentrations of the reagents. Detecting the watermarking agents may allow the identification of the reagent and the concentration of the reagent.

Disclosed herein is method of detecting a watermark and performing a nucleic acid polymerization reaction. Incorporating at least one detectable watermarking agent provides a means for quantifying a target nucleic acid in a sample using Polymerase Chain Reaction. Incorporating at least two detectable watermarking agents provides a means for quantifying multiple nucleic acid targets in a sample (i.e., multiplexing) using Polymerase Chain Reaction. Watermarking agents as used to calculate signal amplitude ratios (as a watermark), may be used in probes for the PCR or qPCR reaction. The probes may use the same watermarking agents to both quantify nucleic acids and generate a watermark. Using the same watermarking agents to both quantify nucleic acids and generate a watermark may involve temporally separating the detections, such that a detection occurs and after an initiation of the polymerase reaction.

A particular watermark may be matched or paired with a chemical composition of established identity, such that when the particular watermark is detected, the identity of the chemical composition can be ascertained. The identity of the chemical composition may comprise characteristics of use that are additional to the chemical make-up. The identity of the chemical composition may include information concerning a time, location, source, or origin. For example, the identity of the chemical composition may include, but not limited to information regarding when it was synthesized, where it was synthesized, what entity performed the synthesis and what methods were used to synthesize the chemical composition. In some cases, such as when the chemical composition is a mixture of components, it may be advantageous to have a watermarking agent matched with an individual component of the mixture, thereby allowing individual components of the chemical composition to be tracked or identified.

Identifying watermarking agents or the chemical compositions paired with watermarking agents may comprise using references signals or reference ratios of signal amplitudes. A watermarking agent may be observed under certain conditions in order to generate a reference signal. The reference signal may then be recorded and stored to allow a future use of the reference signal. Detecting the watermarking agent in a chemical composition may overlay the signal over the reference signal to match the signals. Matching the signal and the reference signal may indicate that the watermarking agent is the same watermarking agent that generated the reference signal.

Watermarking may be used to identify or verify the presence of proprietary or quality controlled reagents. For example, a reaction may be performed to produce an assay result and the assay result undergoes scrutiny. The detection of a watermarked reagent may indicate the assay reagents are not of a formulation expected to produce consistent results. This may be the case for reagents that are expired or past a particular shelf life. A specific watermarking agent may be associated with a time of production of reagent and may be used to identify reagents that are past the shelf life. A formulation of reaction reagents may also be performed by a variety of entities or reagent suppliers. The presence of a watermarked reagent may indicate that the formulation is produced by a specific entity or reagent supplier. For example, the presence of a watermarked agent may indicate the reagents are quality controlled and have been approved by a specific entity or supplier.

Watermarking may be used to identify or verify the synthesis of mixture of reagents. A watermarking agent may indexed to or added concurrently with a reagent during a mixture synthesis process. The watermarking agent may indicate that the reagent has been successfully added to the mixture. A detection of watermarking agents at steps in the mixture synthesis may indicate that a reagent may been properly added to the mixture. The lack of watermarking agent signal may indicate that a reagent is not a component of the mixture or has inadvertently been left out of the mixture. A quality control of the synthesis of the reagents may be performed such that the watermarking signal associated with each reagent is detected.

Watermarking agents may be used in methods as disclosed herein. Watermarking agents may produce a detectable signal that may be used to further identify chemical compositions. Identification of a chemical composition may involve indexing a chemical composition with a watermarking agent. For example a specific watermarking agent may be used with a specific chemical composition such that the presence of a watermarking indicates the presence of a chemical composition. Watermarking agents can comprise a combination of “watermarking components” to produce detectable signal. For example, the watermarking agent may require a watermarking component to react with another watermarking component to produce a detectable signal. In some cases, the detectable signal is a loss of a signal. For example, a baseline signal may be observable in the absence of a watermarking agent. Upon introduction of the watermarking signal, the amplitude of the baseline signal may be decreased. In some cases, a particular portion of the baseline signal may be decrease, for example, at a particular wavelength.

A watermark may also be generated using a ratio of concentrations of watermarking agents. The signal of the watermarking agents may be concentration dependent allowing the generation of different watermarks based on the concentration of the watermarking agents. In some cases, the ratio of the signal amplitudes generated by the watermarking agents may be used as a “watermarking agent” such that it may be used to identify the chemical compound. In an example, the watermark may be used to differentiate between substantially similar chemical compounds comprising identical reaction components using unique ratio of distinctive watermarking agents (wherein the resulting ratio of watermarking agents generates a distinctive watermark) (see Table 1); & (2) determining the presence or absence of a chemical composition (e.g., qPCR reaction mixture) in a given reaction based on the detection of distinctive ratio of watermarking agents generates (i.e., watermark).

TABLE 1
Identifying Two Different Chemical Compositions using Dye Ratios

| Individual Reaction Components | Chemical Composition 1 | Chemical Composition 2 |
|---|---|---|
| Reagent A | concentration of 1 μM | concentration of 1 μM |
| Reagent B | concentration of 2 μM | concentration of 2 μM |
| Reagent C | concentration of 10 μM | concentration of 10 μM |
| Dye 1 | concentration of 15 μM | concentration of 30 μM |
| Dye 2 | concentration of 45 μM | concentration of 45 μM |
| Watermark Dye Ratio | 15 μM:45 μM (1:3) | 30 μM:45 μM (2:3) |
| (Dye 1:Dye 2) | | |

Distinct ratios of detectable labels, such as, for example dyes, may be used as a watermark. Here, Chemical Composition 1 and Chemical Composition 2 are identical in their reagent composition, yet the two chemical compositions exhibit dissimilar Watermark Dye Ratios. Chemical Composition 1 has a Watermark Dye Ratio (Dye 1:Dye 2) of 1:3, while Chemical Composition 2 has a Watermark Dye Ratio (Dye 1:Dye 2) of 2:3. When multiplexing using quantitative Polymerase Chain Reaction (qPCR), each distinct dye-label ratio generates a distinctive fluorescence, thereby watermarking the chemical composition. The resulting watermark may be used to differentiate substantially similar chemical compounds comprising identical reaction components. The unique ratios of these watermarking agents can form singular codes that can be read out during any stage of the chemical composition's lifetime. These singular codes can be used to specifically identify the components of the reaction chemistry without observing the action of the chemistry or the products of the chemical reaction they participate in.

Watermarking Agents

Watermarking agents are used methods as described elsewhere herein. Watermarking agents may be configured to produce a detectable signal when subjected to reaction conditions. Watermarking agents may be dyes, fluorescent molecules, chemiluminescent molecules, magnetic particles, electrets structures exhibiting a permanent dipole, or combinations thereof. A watermarking agent may be composed of a combination of components such that a detectable signal is produced. A watermarking agents may be an enzyme, enzyme substrate, metal, liganded metal, nucleic acid. Watermarking agents may be combined together to form signals different from the individual watermarking agents. Watermarking agents may be coupled to another molecule. For example, a watermarking agent may be a dye coupled to a nucleic acid.

Watermarking agents may emit electromagnetic radiation. Watermarking agents may absorb electromagnetic radiation. Watermarking agents may be quenching agents. Quenching agents may absorb electromagnetic radiation such to “quench” or remove a signal from being detected. Examples of quenchers include TAMRA, BHQ-1, BHQ-2, or Dabcy. Watermarking agents may generate magnetic fields, or be attracted, repelled or otherwise perturbed by magnetic fields. Watermarking agents may generate electric or electrostatic fields.

Dyes may generate a colorimetric signal. The colorimetric signal may be visible to the naked human eye. The colorimetric signal may be visible or identified via spectroscopic methods such to analyze the wavelength of light that are transmitted or absorbed by a solution comprising a dye. Dyes may generate a signal without the presence of an excitation wavelength. The dyes may have a distinct or known signature of absorbed or transmitted light. The detection of a dye signature may comprise identifying an amplitude or amplitude of light signal at different wavelengths. The dye signature may comprise a signal at wavelengths that do not overlap with wavelengths that may be generated by reagents in the chemical composition. For example, a qPCR reaction may be performed which result in signals at first wavelength. The dye signature may comprise signals at wavelength that are generally do not overlap or otherwise change the signal at the first wavelength. In some cases, the signals of the reaction and the dye may be simultaneously detected.

Fluorescent molecules may be used as watermarking agents. Fluorescent molecules may be excited at a wavelength at emit light at another wavelength. The fluorescent molecules may be visible to the naked human eye. The fluorescent molecules may visible or identified via spectroscopic methods such to analyze the wavelength of light that are transmitted or absorbed by a solution comprising a fluorescent molecule. The fluorescent molecules may have a distinct or known signature of excitation or emission wavelength of electromagnetic radiation. The detection of a fluorescent molecule signature may comprise identifying an amplitude or amplitudes of signal at different wavelengths. The fluorescent molecule signature may comprise a signal at wavelengths that do not overlap with wavelengths that may be generated by reagents in the chemical composition. In some cases, the excitation wavelength of the molecule may comprise a signal that does not overlap with wavelengths that may be generated by reagents in the chemical composition. In some cases, the signals of the reaction and the fluorescent molecule may be simultaneously detected. Non-limiting examples of fluorescent molecules that may be used as watermarking agents include Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Cy3, Cy5, Texas Red, Fluorescein(FITC), 6-FAM, 5-FAM, HEX, JOE, TAMRA, ROX, BODIPY FL, Pacific Blue, Pacific Green, Coumarin, Oregon Green, Pacific Orange, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), or derivatives thereof.

Chemiluminescent molecules may be used as watermarking agents. Chemiluminescent molecules may react with another molecule and emit light at wavelength. The chemiluminescent molecules may be visible to the naked human eye. The chemiluminescent molecules may visible or identified via spectroscopic methods such to analyze the wavelength of light that is emitted by a solution comprising a chemiluminescent molecule. The chemiluminescent molecules may have a distinct or known signature of emission wavelengths of light. The detection of a chemiluminescent molecule signature may comprise identifying an amplitude or amplitudes of signal at different wavelengths. The chemiluminescent molecule signature may comprise a signal at wavelengths that do not overlap with wavelengths that may be generated by reagents in the chemical composition. In some cases, the signals of the reaction and the chemiluminescent molecule may be simultaneously detected.

Molecules that produce a magnetic field or otherwise repelled or attracted to a magnetic field may be used as a watermarking agent. For example, the magnetic material may be added and the detectable signal may be the detection of a magnetic field. The material may be ferromagnetic and be attracted to a magnetic field. The detectable signal may be, for example, a visible grouping of magnetic material, when a magnetic field is applied. The grouping of magnetic material may be detected via spectroscopic methods that can determine the magnetic qualities of the material in the solution. The use of molecules that produce magnetic field may be detected by the production of electrical fields generated via magnetic flux. For example, the molecules that produce magnetic fields may be moved in a fashion such that a detectable electric field is generated.

Electrets or molecules that have a permanent dipole may be used as a watermarking agent. The electrets may have a permanent dipole that may be detected as an electrostatic field. The electrets may be detected via interaction with a magnetic field. Electrets may be detected using spectroscopic methods to detect electric or electrostatic fields, or may be observed by the observing the effect of fields upon the introduction or application of charged molecules.

In some case, the watermarking agents may be radioactive species such as a radioactive isotope or other radioactive molecule. Detection of the watermark may comprise the observation radioactive counts demonstrating a decay of the radioactive molecule. The decay of a radioactive molecule may be α, β, or γ decay. Non-limiting examples of radioactive species include ¹⁴C, ¹²³I, ¹²⁴I, ¹²⁵I, ¹³¹I, Tc99m, ³⁵S, or ³H.

In some case, watermarking agents may not generate a signal until the addition of the chemical composition. For example, components in the chemical composition may react with or solubilize watermarking agents such to generate a signal. For example, the watermarking agent may be lyophilized and may otherwise be difficult to analyze or detect a specific signal. Upon addition of a solvent, a watermarking agent may be activated. For example, the watermarking agent may be a metal and the addition of a solvent allows the solvation of a metal. Observation of the metal to ligand charge transfer (or ligand to metal charge transfer) may be used as a detectable signal. The addition of reagents to a metal-ligand complex may allow a ligand to be substituted resulting in a different observable signal. The addition of the chemical composition may also comprise a change in oxidation, change in pH, and change in temperature. The watermarking agent may be oxidizable (or reducible) such that change is oxidation-reduction potential of a solution may result in the generation of a signal. In some cases, the watermarking agent may be pH sensitive or temperature sensitive such that a signal is generated or lost upon a change in temperature or pH.

Watermarking agents may be combined to generate signals that result from the combination of watermarking agents. For example, multiple dyes or fluorescent molecules may be combined. The combined watermarking agents may interact with one another. For example, Forster resonance energy transfer or Fluorescent Resonance Energy Transfer (FRET) may be used to generate a signal. In the example of FRET, a first fluorescent molecule may be excited at a specific wavelength and emit at another wavelength. The emission wavelength of the first fluorescent molecule may be used an excitation wavelength of a second fluorescent molecule and result in the emission from the second fluorescent molecule. The emission generated from the second molecule may be used as a watermarking signal for a chemical composition. Combination of watermarking agents may result in interaction that may be detected, which would otherwise not occur when the watermarking agents are used alone. For example, combining electrets and magnetic particles may result in perturbations in electrostatic or magnetic fields that may be observed. Other examples may use quenching agents, such a metal or other molecule that may absorb electromagnetic radiation, to cause a loss of signal at a particular wavelength when combined with a watermarking agent that generates a signal at the same particular wavelength.

Watermarking agents may be a combination of enzymes and enzyme substrates such to generate a signal. For example, horseradish peroxidase (HRP) or alkaline phosphatase (AP) may be combined with a variety of substrates such as, but not limited to ABTS, OPD, TMB, or luminol, such to generate a colorimetric or chemiluminescent signal that may be detected. Additionally a combination of nucleic acids, and oligonucleotide probes may be used to perform an amplification or hybridization reaction to generate a signal. In some cases, the template, primers, and probe for an amplification reaction may be supplied as a watermarking reagent, such that upon application of reaction conditions, the amplification reaction may generate a signal.

In some cases, a combination of watermarking agents may be used and the resulting ratio of signals generated is used as a unique watermark. The ratio of signal amplitudes may be observed and be used to distinguish between the same combination of watermarking agents, but in a different ratio. This may allow an increase in the potential amount of unique watermarks that exist from a set number of watermarking agents. As a ratio 1:1 vs 1:2 of a first and second watermark may be detected as different signals, the same watermarking agents may be used to identify a plurality of different chemical composition. The ratio may be compared to a reference ratio similarly to as a watermarking agent signal is compared to a reference watermarking signal as described elsewhere herein.

Detection of Watermarking Agents

In aspects as disclosed elsewhere herein, watermarking agents may generate a variety of signals. The detectable signals may be electromagnetic radiation, such as light. The electromagnetic radiation may be detected via fluorimeters, spectrophotometers, or other instruments configured to detect the amount of light or other electromagnetic radiation that passes through a detector. The detection may comprise excitation of the watermarked composition using a radiation source, and the detection of radiation in a different axis from the excitation. The detection may comprise application of radiation to watermarked composition using a radiation source, and the detection of radiation in the same axis. The detectors may involve use of a monochrometer. The detectors may comprise diffraction of detected light in order to detect a particular wavelength. The detectors may detect a plurality of wavelength in a parallel or sequential detection.

Detection may involve the use of detectors for magnetic or electric fields. The detectors may comprise inducing the production of a magnetic or electric field and using an indicator to determine the strength of the induced field. For example, the magnetic field flux generated by the watermarking agent may be detected by an induced electrical field which may detected as a change in voltage.

Detection of watermarking agents comprising radioactivity may be detected via the use of scintillation counters, Geiger counters, dosimeters, or exposure to film. The decayed particles may react with the detectors such to that the particles may be detected or otherwise quantified. The detector may be sensitive between different forms of radioactive decay. The detector may not be sensitive between different forms of radioactive decay.

In cases where there is more than one watermarking agent in the chemical composition, the detection of marking agents may be performed in parallel or in sequence. The same detector may be used for the more than one watermarking agents, or a different detectors may be used depending on the watermarking agent.

In various aspects as disclosed herein, time stamps and geo tags are used. The time stamps and geo-tags may be obtained during a reaction. The time stamps and geo-tags may be obtained during watermarking. The time stamps and geo-tags may be obtained during synthesis of the chemical compositions. The time stamps and geo-tags may be use to calculate an age a mixture or batch of mixture, track the location of a mixture or batch during shipments or use, or be used otherwise to identify a time and place of the watermarking, or watermarked chemical composition.

A report may be outputted compiling the information relating to time stamps, geotags, and watermarking agents. The report may indicate the watermarking agent. The report may indicate the chemical composition. The report may indicate a time of detection of a watermarking agent. The report may indicate a location of detection of a watermarking agent.

Identifying Two Different Chemical Compositions Using a Dye Ratio Watermark (Differentiation Between Analogous Chemical Compounds)

Described herein, in some aspects, is a method of differentiating between analogous chemical compounds. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The mixture may further comprise other reagents (e.g., amplification reagents) including, for example, oligonucleotide primers, dNTPs, a nucleic acid enzyme (e.g., a polymerase), and salts (e.g., Ca²⁺, Mg²⁺, etc.). Next, the mixture be used in a quantitative Polymerase Chain Reaction, whereby a plurality of signals may be generated. The plurality of signals may be detectable in one color channel. The plurality of signals may be detectable in multiple color channels. At least one signal of the plurality of signals may correspond with the presence of a unique combination of two or more of the plurality of nucleic acid molecules. For example, one signal may correspond to the presence of two nucleic acid molecules (e.g., two copies of a nucleic acid sequence). Based on the detecting, the nucleic acid target in the sample may be quantified.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

Quantifying the nucleic acid targets may comprise determining a ratio of a first nucleic acid target to a second nucleic acid target in the sample (e.g., the quantity of the first nucleic acid target relative to the quantity of the second nucleic acid target in the sample). Quantifying the first and second nucleic acid targets may comprise determining an absolute quantity of the first and second nucleic acid targets in the sample. Quantifying the first and second nucleic acid targets may comprise determining a relative quantity of the first and second nucleic acid targets in the sample.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.). A nucleic acid target may be a nucleic acid from a pathogen (e.g., virus, bacteria, etc.). A nucleic acid target may be a nucleic acid suspected of comprising one or more mutations.

### Identifying a Reaction Mixture Using a Dye Ratio Watermark

Described herein, in some aspects, is a method of identifying qPCR reaction mixtures using watermarks. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The mixture may further comprise other reagents (e.g., amplification reagents) including, for example, oligonucleotide primers, dNTPs, a nucleic acid enzyme (e.g., a polymerase), and salts (e.g., Ca²⁺, Mg²⁺, etc.). Next, the mixture be used in a quantitative Polymerase Chain Reaction, whereby a plurality of signals may be generated. The plurality of signals may be detectable in one color channel. The plurality of signals may be detectable in multiple color channels. At least one signal of the plurality of signals may correspond with the presence of a unique combination of two or more of the plurality of nucleic acid molecules. For example, one signal may correspond to the presence of two nucleic acid molecules (e.g., two copies of a nucleic acid sequence). Based on the detecting, the nucleic acid target in the sample may be quantified.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

Quantifying the nucleic acid targets may comprise determining a ratio of a first nucleic acid target to a second nucleic acid target in the sample (e.g., the quantity of the first nucleic acid target relative to the quantity of the second nucleic acid target in the sample). Quantifying the first and second nucleic acid targets may comprise determining an absolute quantity of the first and second nucleic acid targets in the sample. Quantifying the first and second nucleic acid targets may comprise determining a relative quantity of the first and second nucleic acid targets in the sample.

Identifying a given reaction mixture may be generated by assigning the ratio of a first nucleic acid target to a second nucleic acid target in the sample (e.g., the quantity of the first nucleic acid target relative to the quantity of the second nucleic acid target in the sample) to a single chemical composition. As such, the presence or absence of said single chemical composition in a given reaction based on the detection of the ratio of detectable watermarking agents.

### Determining a Ratio

Described herein, in some aspects, is a method of determining a quantify of a first target nucleic acid relative to a quantity of a second target nucleic acid in a sample. First, a mixture may be provided comprising a first plurality of nucleic acid molecules and a second plurality of nucleic acid molecules. The first plurality of nucleic acid molecules may be derived from, and/or may correspond with, the first nucleic acid target in the sample. The second plurality of nucleic acid molecules may be derived from, and/or may correspond with, the second nucleic acid target in the sample. In addition to the first and second pluralities of nucleic acid molecules, other reagents (e.g., amplification reagents) may be provided in the mixture, including, for example, oligonucleotide primers, oligonucleotide probes, dNTPs, a nucleic acid enzyme (e.g., a polymerase), and salts (e.g., Ca²⁺, Mg²⁺, etc.). Next, the first plurality of nucleic acid molecules and the second plurality of nucleic acid molecules may be amplified, thereby generating a plurality of signals. The plurality of signals may be detectable in one color channel. The plurality of signals may be detectable in multiple color channels. Next, the plurality of signals may be detected. The plurality of signals may be detected in a single color channel. The plurality of signals may be detected in multiple color channels. Next, based on the detecting, a ratio may be determined which is representative of a quantity of the first target nucleic acid relative to a quantity of the second target nucleic acid in the sample. The method may not include a step of quantifying the first plurality of nucleic acid molecules or the second plurality of nucleic acid molecules. The first target nucleic acid and the second target nucleic acid in the sample may be quantified based on the ratio.

The plurality of signals may be generated from the plurality of oligonucleotide probes. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. Nucleic acid amplification may release a signal tag from the plurality of probes, thereby generating the plurality of signals. A plurality of signals may be a plurality of fluorescent signals, a plurality of chemiluminescent signals, or a combination thereof.

A ratio may be representative of a relative abundance of target nucleic acids following completion of a qPCR reaction. Similarly, a ratio may be representative of a qPCR chemical reaction.

The first plurality of nucleic acid molecules may be copies of the first nucleic acid target, where the copies have been transferred from the sample into the mixture. The second plurality of nucleic acid molecules may be copies of the second nucleic acid target, where the copies have been transferred from the sample into the mixture. The first plurality of nucleic acid molecules and the second plurality of nucleic acid molecules may originate from the sample. The first plurality of nucleic acid molecules may be products of nucleic acid amplification (e.g., PCR) of the first target nucleic acid. The second plurality of nucleic acid molecules may be products of nucleic acid amplification (e.g., PCR) of the second target nucleic acid. The first plurality of nucleic acid molecules may be products of nucleic acid extension of the first target nucleic acid. The second plurality of nucleic acid molecules may be products of nucleic acid extension of the second target nucleic acid. The first plurality of nucleic acid molecules may be products of reverse transcription of the first target nucleic acid. The second plurality of nucleic acid molecules may be products of nucleic acid extension of the second target nucleic acid.

### Assays

In some cases, assays may be run using the reagents in the chemical composition. Assay may use a reagent to perform a reaction. The reaction may comprise a hybridization reaction. For example, the reagent may comprise a nucleic acid and hybridize with another nucleic acid. The nucleic acid and the another nucleic acid may be complementary to one another. The reaction may comprise an extension reaction. For example, the reaction may comprise extending a nucleic molecule by the addition of a nucleotide. The reaction may comprise a polymerase chain reaction.

Any number of nucleic acid targets may be detected using assays of the present disclosure. In some cases, an assay may unambiguously detect at least 3, 4, 5, 6, 7, 8, 9, 10, 11, 12, 13, 14, 15, 20, 30, 40, 50 nucleic acid targets, or more. In some cases, an assay may unambiguously detect at most 50, 40, 30, 20, 15, 14, 13, 12, 11, 10, 9, 8, 7, 6, 5, 4, or 3 nucleic acid targets. An assay may comprise any number of reactions, where the results of the reactions together identify a plurality of nucleic acid targets, in any combination of presence or absence. An assay may comprise 2, 3, 4, 5, 6, 7, 8, 9, 10 reactions, or more. Each reaction may be individually incapable of non-degenerately detecting the presence or absence of any combination of nucleic acid targets. However, the results of each reaction together may unambiguously detect the presence or absence of each of the nucleic acid targets.

Reactions may be performed in the same sample solution volume. For example, a first reaction may generate a fluorescent signal in a first color channel, while a second reaction may generate a fluorescent signal in a second color channel, thereby generating two measurements for comparison. Alternatively, reactions may be performed in different sample solution volumes. For example, a first reaction may be performed in a first sample solution volume and generate a fluorescent signal in a given color channel, and a second reaction may be performed in a second sample solution volume and generate a fluorescent signal in the same color channel or a different color channel, thereby generating two measurements for comparison.

Assay reactions as described herein may be conducted in parallel. In general, parallel assay reactions are reactions that occur in the same reaction vessel and at the same time. For example, parallel nucleic acid amplification reactions may be conducted by including reagents used for each nucleic acid amplification reaction in a reaction vessel to obtain a reaction mixture and subjecting the reaction mixture to conditions used for each nucleic acid amplification reaction. Any suitable number of nucleic acid amplification reactions may be conducted in parallel. In some cases, at least 1, 2, 3, 4, 5, 6, 7, 8, 9, 10, 11, 12, 13, 14, 15, 16, 17, 18, 19, 20, or more nucleic acid amplification reactions are conducted in parallel.

Each oligonucleotide probe may be labeled with a fluorophore. The fluorophores may be capable of being detected in a single optical channel. For example, the fluorophores may each comprise similar emission wavelength spectra, such that they can be detected in a single optical channel.

### Amplification

In some aspects, the disclosed methods comprise nucleic acid amplification. Amplification conditions may comprise thermal cycling conditions, including temperature and length in time of each thermal cycle. The use of particular amplification conditions may serve to modify the signal intensity of each signal, thereby enabling each signal to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and a RNA molecule may be generated. In another example, a RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonuclotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Nucleic Acid Targets

A nucleic acid target of the present disclosure may be derived from a biological sample. A biological sample may be a sample derived from a subject. A biological sample may comprise any number of macromolecules, for example, cellular macromolecules. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may comprise one or more cells. A biological sample may be, for example, blood, plasma, serum, urine, saliva, mucosal excretions, sputum, stool or tears.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses) in a subject.

Nucleic acid targets may be of various concentrations in the reaction. The nucleic acid sample may be diluted or concentrated to achieve different concentrations of nucleic acids. The concentration of the nucleic acids in the nucleic acid sample may at least 0.1 nanograms per microliter (ng/μL), 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or more. In some cases, the concentration of the nucleic acids in the nucleic acid sample may be at most ng/μL, 0.2 ng/μL, 0.5 ng/μL, 1 ng/μL, 2 ng/μL, 3 ng/μL, 5 ng/μL, 10 ng/μL, 20 ng/μL, 30 ng/μL, 40, ng/μL, 50 ng/μL, 100 ng/μL, 1000 ng/μL, 10000 ng/μL or less.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase generally refers to an enzyme that is capable of incorporating nucleotides to a strand of DNA in a template bound fashion. A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exo activity and degrade a probe resulting in a detectable signal. A nucleic acid enzyme may be capable, under appropriate conditions, of releasing a quencher from an oligonucleotide probe.

### Reactions

In various aspects disclosed elsewhere herein, reactions are performed. A reaction may comprise contacting nucleic acid targets with one or more oligonucleotide probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

### Oligonucleotide Primers

In various aspects disclosed elsewhere herein, oligonucleotide primers are used. An oligonucleotide primer (or “amplification oligomer”) of the present disclosure may be a deoxyribonucleic acid. An oligonucleotide primer may be a ribonucleic acid. An oligonucleotide primer may comprise one or more non-natural nucleotides. A non-natural nucleotide may be, for example, deoxyinosine.

An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences.

A mixture may comprise a plurality of forward oligonucleotide primers. A plurality of forward oligonucleotide primers may be a deoxyribonucleic acid. Alternatively, a plurality of forward oligonucleotide primers may be a ribonucleic acid. A plurality of forward oligonucleotide primers may be between about 5 and about 50 nucleotides in length. A plurality of forward oligonucleotide primer may be at least 5, 10, 11, 12, 13, 14, 15, 16, 17, 18, 19, 20, 25, 30, 35, 40, 45, or 50 base pairs in length, or more. A plurality of forward oligonucleotide primer may be at most 50, 45, 40, 35, 30, 25, 20, 19, 18, 17, 16, 15, 14, 13, 12, 11, 10, or 5 nucleotides in length.

A mixture may comprise a plurality of reverse oligonucleotide primers. A plurality of reverse oligonucleotide primers may be a deoxyribonucleic acid. Alternatively, a plurality of reverse oligonucleotide primers may be a ribonucleic acid. A plurality of reverse oligonucleotide primers may be between about 5 and about 50 nucleotides in length. A plurality of reverse oligonucleotide primer may be at least 5, 10, 11, 12, 13, 14, 15, 16, 17, 18, 19, 20, 25, 30, 35, 40, 45, or 50 base pairs in length, or more. A plurality of reverse oligonucleotide primer may be at most 50, 45, 40, 35, 30, 25, 20, 19, 18, 17, 16, 15, 14, 13, 12, 11, 10, or 5 nucleotides in length.

In some aspects, a mixture may include one or more synthetic (or otherwise generated to be different from the target of interest) primers for PCR reactions.

In some aspects, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule. In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

### Oligonucleotide Probes

In various aspects disclosed elsewhere herein, oligonucleotide probes are used. Samples, mixtures, kits, and compositions of the present disclosure may comprise an oligonucleotide probe, also referenced herein as a “detection probe” or “probe”. An oligonucleotide probe may be a nucleic acid (e.g., DNA, RNA, etc.). An oligonucleotide probe may comprise a region complementary to a region of a nucleic acid target. The concentration of an oligonucleotide probe may be such that it is in excess relative to other components in a sample.

An oligonucleotide probe may comprise a non-target-hybridizing sequence. A non-target-hybridizing sequence may be a sequence which is not complementary to any region of a nucleic acid target sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

A sample may comprise more than one oligonucleotide probe. Multiple oligonucleotide probes may be the same, or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be a nucleic acid (e.g., DNA, RNA, etc.). An oligonucleotide probe may be at least 2, 3, 4, 5, 6, 7, 8, 9, 10, 20, 30, 40, or 50 nucleotides in length, or more. An oligonucleotide probe may be at most 50, 40, 30, 20, 10, 9, 8, 7, 6, 5, 4, 3, or 2 nucleotides in length.

In some cases, 2, 3, 4, 5, 6, 7, 8, 9, 10, 11, 12, 13, 14, 15, 20, or more different oligonucleotide probes may be used. Each oligonucleotide probe may correspond to (e.g., capable of binding to) a given region of a nucleic acid target (e.g., a chromosome) in a sample. In one example, a first oligonucleotide probe is specific for a first region of a first nucleic acid target, a second oligonucleotide probe is specific for a second region of the first nucleic acid target, and a third oligonucleotide probe is specific for a third region of the first nucleic acid target. Each oligonucleotide probe may comprise a signal tag with about equal emission wavelengths. In some cases, each oligonucleotide probe comprises an identical fluorophore. In some cases, each oligonucleotide probe comprises a different fluorophore, where each fluorophore is capable of being detected in a single optical channel.

A probe may correspond to a region of a nucleic acid target. For example, a probe may have complementarity and/or homology to a region of a nucleic acid target. A probe may comprise a region which is complementary or homologous to a region of a nucleic acid target. A probe corresponding to a region of a nucleic acid target may be capable of binding to the region of the nucleic acid target under appropriate conditions (e.g., temperature conditions, buffer conditions. etc). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A probe may be a nucleic acid complementary to a region of a given nucleic acid target. Each probe used in the methods and assays of the presence disclosure may comprise at least one fluorophore. A fluorophore may be selected from any number of fluorophores. A fluorophore may be selected from three, four, five, six, seven, eight, nine, or ten fluorophores, or more. One or more oligonucleotide probes used in a single reaction may comprise the same fluorophore. In some cases, all oligonucleotide probes used in a single reaction comprise the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

An oligonucleotide probe may comprise a detectable label. A detectable label may have the same molecular structure as a watermarking agent. A detectable label may be a chemiluminescent label. A detectable label may comprise a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a mixture.

A reaction may generate one or more signals. A reaction may generate a cumulative intensity signal comprising a sum of multiple signals. A signal may be a chemiluminescent signal. A signal may be a fluorescent signal. A signal may be generated by an oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more oligonucleotide probes. The number of signals generated in an assay may correspond to the number of oligonucleotide probes and nucleic acid targets present.

N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths. A range of wavelengths may be referred to as a “channel,” “color channel,” or “optical channel.”

The presence or absence of one or more signals may be detected. One signal may be detected, or multiple signals may be detected. Multiple signals may be detected simultaneously. Alternatively, multiple signals may be detected sequentially. The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules.

### Kits

The present disclosure also provides kits for watermark analysis. Kits may comprise one or more oligonucleotide probes. Oligonucleotide probes may be lyophilized. Different oligonucleotide probes may be present at different concentrations in a kit. Oligonucleotide probes may comprise a fluorophore and/or one or more quenchers.

Kits may comprise one or more sets of oligonucleotide primers (or “amplification oligomers”) as described herein. A set of oligonucleotide primers may comprise paired oligonucleotide primers. Paired oligonucleotide primers may comprise a forward oligonucleotide primer and a reverse oligonucleotide primer. A set of oligonucleotide primers may be configured to amplify a nucleic acid sequence corresponding to particular targets. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence. Different sets of oligonucleotide primers may be configured to amplify nucleic acid sequences. In one example, a first set of oligonucleotide primers may be configured to amplify a first nucleic acid sequence, and a second set of oligonucleotide primers may be configured to amplify a second nucleic acid sequence. Oligonucleotide primers configured to amplify nucleic acid molecules may be used in performing the disclosed methods. In some cases, all of the oligonucleotide primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. Kits may comprise instructions for using any of the foregoing in the methods described herein.

Kits provided herein may be useful in, for example, calculating at least first and second sums, each being a sum of multiple target signals corresponding with a first and second target nucleic acid.

### EXAMPLES

### Example 1—Watermarking Multiplexed Quantitative PCR

Multiplexed quantitative Polymerase Chain Reaction (qPCR) reaction chemistry generally consists of a mixture of detection probes, amplification oligomers, and polymerase enzymes. Commonly, the same qPCR reaction mixture is added to distinct, heterogeneous biological samples. The resulting multiplexing reaction mixture produces a fluorescence signature that identifies whether or not the target nucleic acid sequence is present in the corresponding sample. Yet, no signature is generated to identify the reaction mixture itself. Thus, the use of a detectable label provides a novel means to independently identify the chemical composition (qPCR reaction mixture) itself. Table 2 illustrates the ability to use watermarking to differentiate between individual reaction components within a chemical composition in a 2-channel qPCR experiment.

TABLE 2
Differentiation Between Individual Components Within
a Chemical Composition Using Watermarking in a
2-Channel Quantitative PCR (qPCR) Experiment.

| Individual Components within | | |
|---|---|---|
| a Chemical Composition | Concentration | |
| FWD Primer A | 100 | nM |
| RWD Primer A | 100 | nM |
| FWD Primer B | 100 | nM |
| RWD Primer B | 100 | nM |
| Fluorescent detection probe | 50 | nM |
| specific to Target A with Dye 1 | | |
| Fluorescent detection probe | 50 | nM |
| specific to Target B with Dye 2 | | |
| DNA polymerase | | |
| Free Dye 1 | 10 | nM |
| Free Dye 2 | 30 | nM |
| Target A | 10 | fM |
| Target B | 100 | aM |

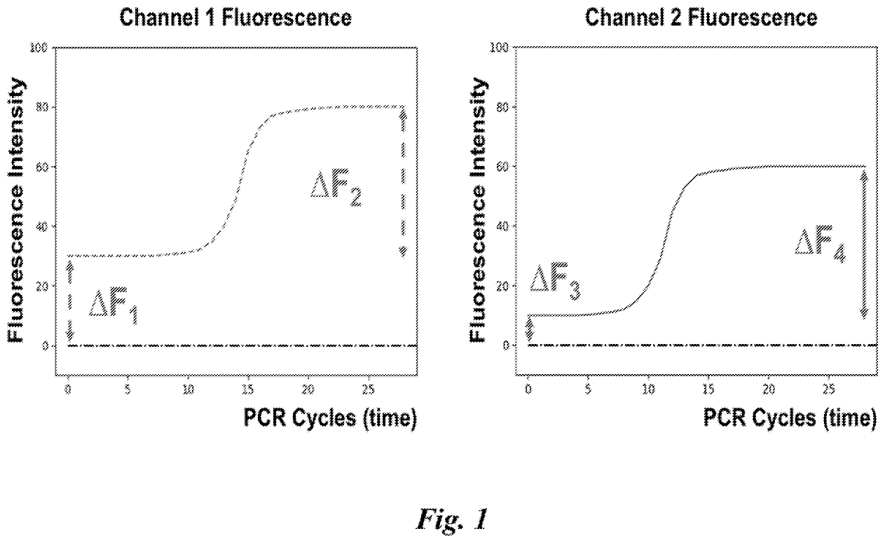

Free Dye 1 and Free Dye 2 are used as distinct labels for the purpose of identifying the individual reaction components within a chemical composition. At the end of each cycle in a qPCR amplification experiment, Free Dye 1 and Free Dye 2 generate unique fluorescent signatures correlating to the relative abundance and/or presence of Target A and Target B and/or amplicons of Target A and Target B. Additionally, the qPCR reaction mixture itself generates a unique fluorescence signature (i.e. watermark). See FIG. 1.

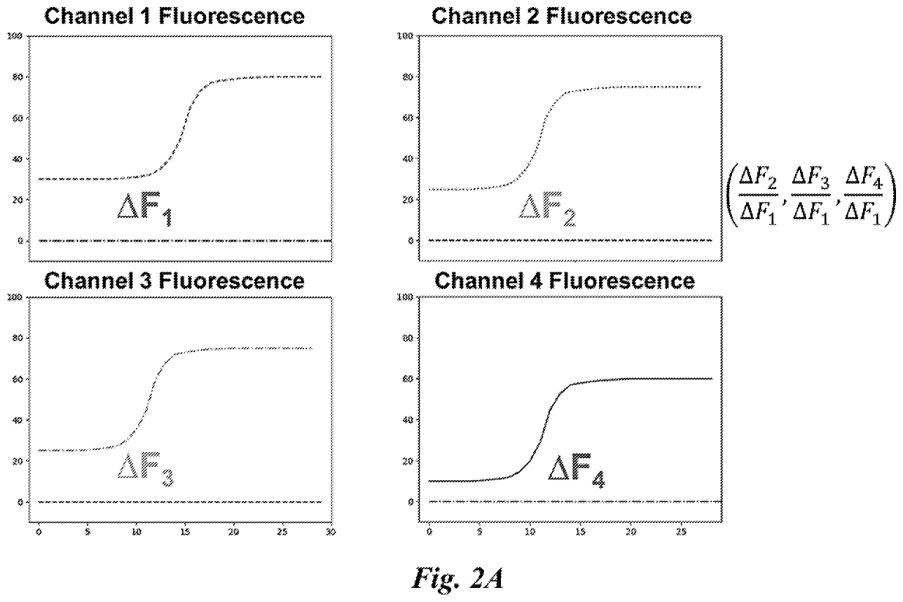

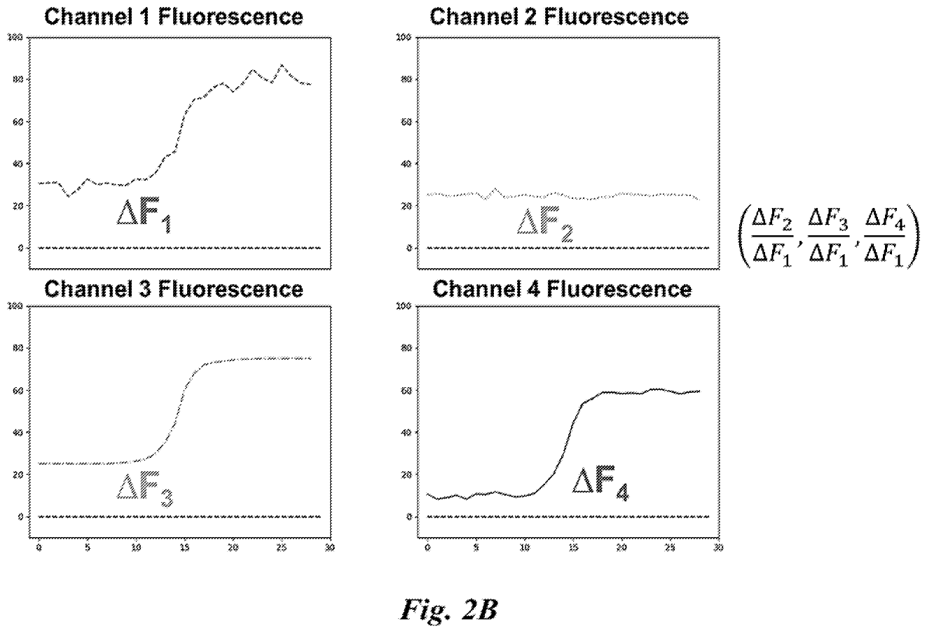

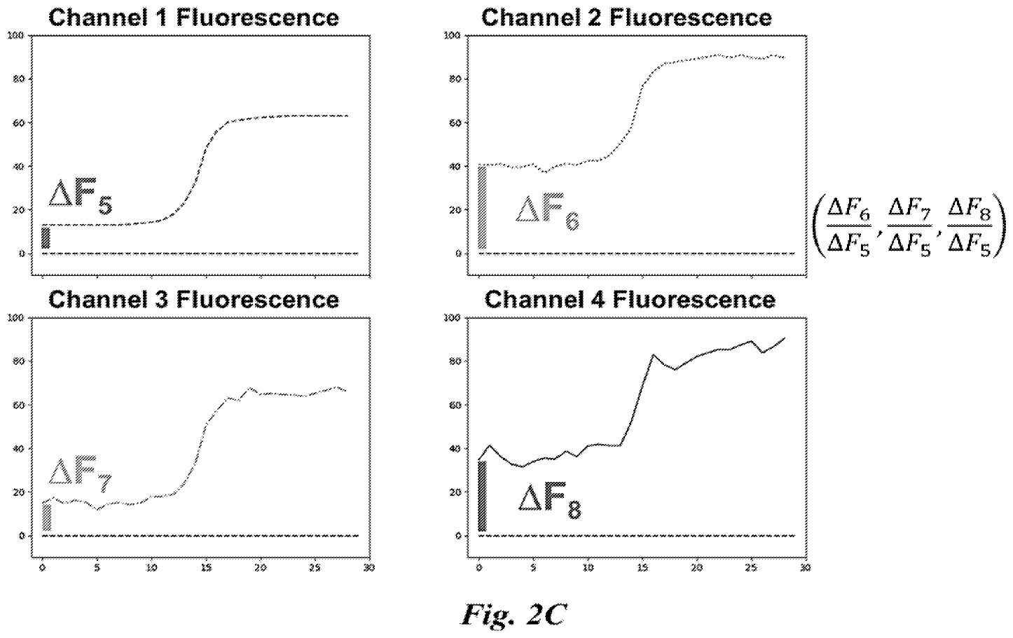

FIG. 1 shows the initial fluorescence above the noise threshold (ΔF1), is generated by the initial fluorescence in channel 1. ΔF2 is generated by the amplification and identification of Target A and the action of the fluorescent detection probe specific for Target A. The ratio ΔF2/ΔF1 can be used to identify the qPCR reaction in channel 1. Similarly, the initial fluorescence above the noise threshold (ΔF3), represents the initial fluorescence in channel 2. ΔF4 is generated by the amplification and identification of Target B and the action of the fluorescent detection probe specific for Target B. The ratio of ΔF4/ΔF3 can be used to identify the qPCR reaction in channel 2. The dye ratio of ΔF3/ΔF1 represents the watermark of the chemical composition itself, and can be used, for example, to identify a particular qPCR reaction from any another qPCR reaction mixture, while serving as an indicator as to whether additional chemical component have been added to the chemical composition. The same principle can be extended to four-channel qPCR instrumentation (See FIG. 2A).

FIG. 2A shows a four-color qPCR experiment. Here, the reaction mixture watermark is identified by a unique combination of three ratios: ΔF2/ΔF1, ΔF3/ΔF1, ΔF4/ΔF1. By using a watermark, the actual qPCR curves themselves are not needed to identify the qPCR reaction composition. When the qPCR reaction mixture is run with a separate set of samples (See FIG. 2B), it generates a different set of qPCR curves, but retains the same signature watermark: ΔF2/ΔF1, ΔF3/ΔF1, ΔF4/ΔF1.

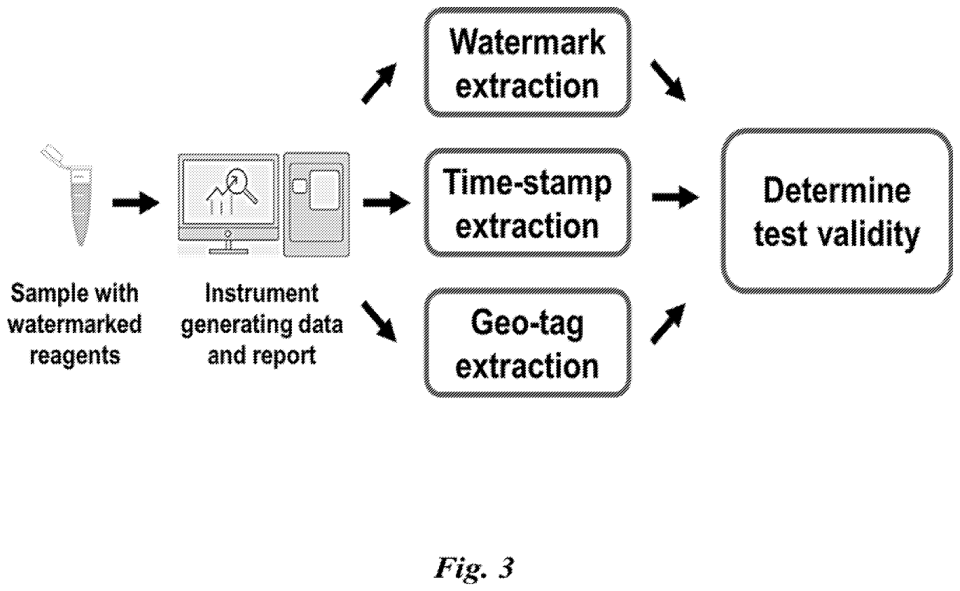

FIG. 2B shows the qPCR reaction mixture is identifiable by its signature watermark: ΔF2/ΔF1, ΔF3/ΔF1, ΔF4/ΔF1, even when used in combination with different target samples. Relatedly, other compositions, as shown in FIG. 2C, may be engineered to generate a distinct fluorescence watermarks, thereby providing a novel means to identify various qPCR reaction mixtures. For example, ΔF6/ΔF5, ΔF7/ΔF5, ΔF8/ΔF5 generates a unique watermark from that of ΔF2/ΔF1, ΔF3/ΔF1, ΔF4/ΔF1.

Where a watermarked qPCR composition (with a known manufacturing date) is run on standard qPCR instrumentation, the watermark, combined with the time-stamp of when the assay was run, and location of where the instrument was located, may be used to determine whether to report the result (i.e. validating the qPCR itself). See FIG. 3. This could, for example, prevent operators and clinicians from running diagnostic qPCR assays past their expiration date, or in regions where regulatory approval has not yet been received.

FIG. 3 shows watermarked qPCR composition with a known manufacturing date are run on standard qPCR instrumentation. The combination of the watermark, combined with the time-stamp of when the assay was run, and location of where the instrument was located, may be used to determine whether to report the result of the qPCR itself. In one embodiment, the watermark allows for monitoring and tracking chemical compositions through its lifetime; in another embodiment, the watermark provides a means for verification as to when and where a chemical composition was manufactured; in another embodiment, the watermark provides a means for certifying a chemical composition has not passed it designed expiration; in another embodiment, the watermark provides a means for authenticating a chemical compositions have not impermissibly entered regions where the chemistry has not been approved for use (e.g., traceability of pharmaceutical compounds).

### Example 2: Watermarking a Chemical Composition and Using a Watermark

A chemical composition is obtained containing a mixture of molecules. The identity of the molecules and the concentration of the molecules are known. The identity of the molecules and concentration are recorded for reference. A watermarking agent is added into the composition, reaction conditions are applied to the composition, and a signal generated by the watermark under such conditions is observed. The observed signal is recorded and indexed with the record of the identity of the molecules and concentration of the molecules of the chemical composition. The indexing of watermarking agents to their respective chemical compositions are stored in record book with a collection of reference signals corresponding to each watermarking agent. At a later point in time the chemical composition is transferred in which the identity and concentration of the molecules are no longer certain. The watermark may be detected and compared to the records of references signals and matched with a reference signal. Upon matching the reference signal to the watermarking agent signal, the watermarking agent that is in the chemical composition is identified. Using the indexed records, the chemical composition that is paired with the watermarking agent is determined, allowing the chemical compositions identity and the concentration of the molecules to be determined.

### Example 3: Combined Watermarking Agents as a Watermark

A chemical composition is obtained containing a mixture of molecules. The identity of the molecules and the concentration of the molecules are known. The identity of the molecules and concentration are recorded for reference. Two watermarking agent are added into the composition and the signal of the combined watermarking agents is observed. The observed signal is recorded and indexed with the record of the identity of the molecules and concentration of the molecules of the chemical composition. The signal resulting from the combined watermarking agents is indexed to the chemical compositions and is stored in record book with a collection of reference signals corresponding to each combination of watermarking agents. At a later point in time the chemical composition is transferred in which the identity and concentration of the molecules are no longer certain. The signal is detected and compared to the records of references signals and matched with a reference signal. Upon matching the reference signal to the combined watermarking agent signal, the combination of watermarking agents that is in the chemical composition is identified. Using the indexed records, the chemical composition that is paired with the combined watermarking agents is determined, allowing the chemical compositions identity and the concentration of the molecules to be determined.

### Example 4: Verifying Test Results Using a Geo-Tag and a Time Stamp

An assay is run using a reagent mixture and the result in processed to determine a test result. The test result does not match up with previously ran tests on using a similar reagent mixture and a similar template. The mixture containing the test reagents is subjected to detection methods to identify watermarking agents. A watermarking signal is detected and a time stamp and a geo-tag is associated with this test result. FIG. 3 shows a schematic of the test. The watermarking signal that is detected indicates that this reagent mixture is synthesized at a certain time and location. Using the time stamp and geo-tag the reagent may be determined to be in a location that is distant from the synthesis location and may have degraded due to the shipping time. A geo-tag on the test result may also indicate the test was performed in a location that the test reagents have not been approved in. Additionally the time stamp may indicate that the reagent is past the recommended shelf life. The lack of a watermarking agent may also reveal that the test reagents are not from the certain supplier that watermarks the reagents and may be of a lesser or inferior quality.

What is claimed is:

1. A method of verifying the synthesis of a chemical mixture, said method comprising
a) providing a sample comprising:
i) a chemical mixture, comprising a first chemical component and a second chemical component;
ii) a first watermarking agent configured to emit a first detectable signal, wherein said first watermarking agent indicates the presence of said first chemical component; and
iii) a second watermarking agent configured to emit a second detectable signal, wherein said second watermarking agent indicates the presence of said second chemical component; and
b) detecting the presence of said first detectable signal and said second detectable signal, thereby verifying the synthesis of said chemical composition.

2. The method of claim 1, further comprising obtaining a time stamp.

3. The method of claim 2, wherein said time stamp indicates a synthesis time.

4. The method of claim 3, further comprising outputting a report with a value of said first detectable signal, a value of said detectable signal, and said time stamp.

5. The method of claim 1, further comprising obtaining a geo-tag.

6. The method of claim 5, wherein said geo-tag indicates a location of verification of synthesis or a location of synthesis.

7. A method of tracking a batch of chemical mixture, said method comprising
a) providing chemical mixture comprising:
i) a chemical mixture; and
ii) a first watermarking agent configured to emit a first detectable signal
b) detecting said first detectable signal; and
c) comparing said first detectable signal to a reference signal indicative of a batch of a chemical mixture.

8. The method of claim 7, wherein the sample further comprises a second watermarking agent configured to emit a second detectable signal.

9. The method of claim 8, further comprising calculating a ratio of said first detectable signal and said second detectable signal.

10. The method of claim 9, further comprising comparing said ratio to a reference ratio indicative of a batch of a chemical mixture.

11. The method of claim 7, further comprising obtaining a geo-tag.

12. The method of claim 11, further comprising outputting a report indicative of the presence of said batch at a location indicated by said geo-tag.

13. The method of claim 7, further comprising obtaining a time stamp.

14. The method of claim 13, wherein said reference signal is indicative of a time of manufacture of said batch.

15. The method of claim 14, further comprising outputting a report.

16. The method of claim 15, wherein said report indicates the presence of a batch at a time of said time stamp.

17. The method of claim 16, wherein said report indicates an age of said batch.

18. A method of watermarking a chemical composition, the method comprising:
a) obtaining a chemical composition; and
b) introducing into said chemical composition a watermarking agent that is configured to produce a detectable signal upon application of reaction conditions to the chemical composition, the presence of said detectable signal indicating that the chemical composition has been watermarked.

19. The method of claim 18, wherein said detectable signal is dependent on a concentration of said watermarking agent.

20. The method of claim 18, wherein said detectable signal is configured to be compared with or compared to a reference signal.

21. The method of claim 18, wherein said watermarking agent is a selected from the group consisting of: (a) a dye, (b) a fluorescent molecule, (c) a chemiluminescent label, (d) a magnetic particle, (e) an electret structure exhibiting a permanent dipole, (f) a radioactive species, and any combination thereof.

22. The method of claim 18, wherein said watermarking agent absorbs electromagnetic radiation.

23. The method of claim 18, wherein said watermarking agent emits electromagnetic radiation.

24. The method of claim 18, wherein said watermarking agent comprises a quenching agent.

25. The method of claim 18, wherein said watermarking agent comprises a metal.

26. The method of claim 18, wherein said watermarking agent comprises a nucleic acid.

27. The method of claim 18, wherein said watermarking agent comprises an enzyme.

28. The method of claim 18, wherein said watermarking agent is an enzyme substrate.

29. The method of claim 18, wherein said watermarking agent is an enzyme substrate configured to be processed to produce said detectable signal.

30. The method of claim 18, wherein said watermarking agent comprises one or more components for performing a reaction that produces said detectable signal.

31. The method of claim 30, wherein said components comprise nucleic acids and said reaction comprises a nucleic acid extension.

32. The method of claim 18, wherein said watermarking agent is configured to produce said detectable signal upon solvation of said watermarking agent.

33. The method of claim 18, wherein said detectable signal comprises electromagnetic radiation.

34. The method of claim 33, wherein said electromagnetic radiation comprises light.

35. The method of claim 18, wherein said detectable signal comprises electromagnetic radiation at a particular wavelength.

36. The method of claim 18, wherein said detectable signal comprises electromagnetic radiation at a plurality of wavelengths.

37. The method of claim 18, wherein said detectable signal comprises a magnetic field.

38. The method of claim 18, wherein said detectable signal comprises an electric field.

39. The method of claim 18, wherein said detectable signal comprises a radioactive decay.

40. The method of claim 18, wherein said detectable signal comprises a change in signal from a baseline signal.

41. The method of claim 40, wherein said change in signal from a baseline signal comprises a decrease in signal amplitude.

42. The method of claim 18, further comprising introducing a second watermarking agent.

43. The method of claim 42, wherein the combination of said first and second watermarking agents generates said detectable signal upon application of said reaction conditions to the chemical composition.

44. The method of claim 42, wherein said second watermarking agent is configured to produce a second detectable signal upon application of reaction conditions to the chemical composition.

45. The method of claim 42, wherein said chemical composition comprises said watermarking agent at a first concentration and said second watermarking agent at a second concentration.

46. The method of claim 42, wherein said watermarking agent and said second watermarking reagent are configured to produce a detectable signal and a second detectable signal at a ratio of said detectable signal and a second detectable signal; and wherein said ratio indicates that the chemical composition has been watermarked.

47. The method of claim 42, wherein said watermarking agent and said second watermarking agent are configured to be detected simultaneously.

48. The method of claim 42, wherein said watermarking agent and said second watermarking agent are configured to be detected sequentially.

49. The method of claim 42, wherein said watermarking agent and said second watermarking agent are configured to be detected using the same instrument.

50. The method of claim 49, wherein said watermarking agent and said second watermarking agent are configured to be detected using the same fluorescent channel.

51. The method of claim 18, wherein said chemical composition further comprises assay reagent(s).

52. The method of claim 51, wherein said assay reagent(s) comprise reagent(s) for a nucleic acid extension reaction.

53. The method of claim 52, wherein said nucleic acid extension reaction is polymerase chain reaction (PCR).

54. The method of claim 51, wherein said assay reagent(s) comprise a buffer, a salt, or an enzyme.

55. The method of claim 53, wherein said polymerase chain reaction is a quantitative polymerase chain reaction (qPCR).

56. The method of claim 51, wherein said assay reagent(s) comprises a DNA polymerase, a reverse transcriptase, a RNA polymerase, or a combination thereof.

57. The method of claim 51, wherein said assay reagent(s) comprise a dNTP, a salt, a buffer, or a combination thereof.

58. The method of claim 51, wherein said assay reagents(s) comprise an oligonucleotide primer.

59. The method of claim 58, wherein said oligonucleotide primer is configured to target a nucleic acid sequence.

60. The method of claim 58, wherein said oligonucleotide primer is configured to hybridize to a nucleic acid sequence.

61. The method of claim 51, wherein said assay reagent(s) comprise an oligonucleotide probe.

62. The method of claim 61, wherein said oligonucleotide probe is a TaqMan® probe.

63. The method of claim 61, wherein said oligonucleotide probe is a molecular beacon.

64. The method of claim 61, wherein said oligonucleotide probe comprises a label.

65. The method of claim 64, wherein said label and said watermarking agent comprise the same molecular structure.

66. The method of claim 64, wherein said label and said watermarking agent comprise a different molecular structure.

67. The method of claim 64, wherein said label produces said detectable signal.

68. A method of identifying a chemical composition, said method comprising:
a) obtaining a chemical composition comprising:
i) a first watermarking agent configured to emit a first detectable signal; and
ii) a second watermarking agent configured to emit a second detectable signal;
b) detecting said first detectable signal and said second detectable signal;
c) calculating a ratio of said first detectable signal and said second detectable signal; and
d) comparing said ratio to a reference ratio indicative of said chemical composition of chemical reagents, thereby identifying said chemical composition.

69. The method of claim 68, wherein said ratio is dependent on a concentration of said first watermarking agent and a concentration of said second watermarking agent.

70. The method of claim 68, wherein said chemical composition further comprises nucleic acids.

71. The method of claim 68, further comprising performing a nucleic acid extension reaction.

72. The method of claim 71, wherein said extension reaction is a polymerase chain reaction.

73. The method of claim 72, wherein said polymerase chain reaction is a quantitative polymerase chain reaction.

74. The method of claim 68, wherein said chemical composition comprises a mixture of polymerase chain reaction reagents comprising: a buffer, a salt, a dNTP, an enzyme, or a combination thereof.

75. The method of claim 74, wherein said salt comprises magnesium, sodium, potassium, chloride, or citrate.

76. The method of claim 74, wherein said enzyme is selected from the group consisting of DNA polymerase, RNA polymerase, reverse transcriptase.

77. The method of claim 70, wherein said nucleic acids comprise a first set of oligomers configured to amplify a first sequence and a first probe comprising said first watermarking agent configured to emit said first detectable signal upon amplification of said first sequence.

78. The method of claim 77, wherein said first probe is a nucleic acid.

79. The method of claim 78, wherein said first probe hybridizes to at least a portion of said first sequence.

80. The method of claim 77, wherein said first detectable watermarking agent provides a quantitative ratio measurement corresponding to an abundance of a first target sequence in the chemical composition.

81. The method of claim 77, wherein said nucleic acids comprise a second set of oligomers configured to amplify a second sequence and a second probe comprising said second watermarking agent configured to emit said second detectable signal upon amplification of said second sequence.

82. The method of claim 81, wherein said second probe is a nucleic acid.

83. The method of claim 82, wherein said second probe hybridizes to at least a portion of said second sequence.

84. The method of claim 81, wherein said second detectable watermarking agent provides a quantitative ratio measurement corresponding to an abundance of a second target sequence in the chemical composition.

85. The method of claim 77, wherein said first probe is a TaqMan® probe.

86. The method of claim 77, wherein said first probe is a molecular beacon or molecular torch.

87. The method of claim 81, wherein said first probe and said second probe is a TaqMan® probe.

88. The method of claim 81, wherein said first probe and said second probe is a molecular beacon or molecular torch.

89. The method of claim 68, further comprising subsequent to b), detecting at a second time point said first detectable signal and said second detectable signal.

90. The method of claim 89, further comprising subsequent to said second time point, processing said first detectable signal to determine a presence or concentration of said first sequence.

91. The method of claim 90, further comprising subsequent to said second time point, processing said second detectable signal to determine a presence or concentration of said second sequence.

92. The method of claim 68, wherein said chemical composition comprises a chemical reagent.

93. The method of claim 92, wherein said chemical composition comprises two chemical reagents.

94. The method of claim 68, wherein said reaction conditions are polymerase chain reaction (PCR) conditions.

95. A watermarked chemical composition, comprising:
a) one or more chemical components; and
b) a watermarking agent that is configured to produce a detectable signal upon application of reaction conditions to the composition, the presence of said detectable signal indicating that the composition has been watermarked.

96. The watermarked chemical composition of claim 95, wherein said detectable signal is dependent on a concentration of said watermarking agent.

97. The watermarked chemical composition of claim 95, wherein said detectable signal is configured to be compared with or compared to a reference signal.

98. The watermarked chemical composition of claim 95, wherein said watermarking agent is a selected from the group consisting of: (a) a dye, (b) a fluorescent molecule, (c) a chemiluminescent label, (d) a magnetic particle, (e) an electret structure exhibiting a permanent dipole, (f) a radioactive species, and any combination thereof.

99. The watermarked chemical composition of claim 95, wherein said watermarking agent absorbs electromagnetic radiation.

100. The watermarked chemical composition of claim 95, wherein said watermarking agent emits electromagnetic radiation.

101. The watermarked chemical composition of claim 95, wherein said watermarking agent comprises a quenching agent.

102. The watermarked chemical composition of claim 95, wherein said watermarking agent comprises a metal.

103. The watermarked chemical composition of claim 95, wherein said watermarking agent comprises a nucleic acid.

104. The watermarked chemical composition of claim 95, wherein said watermarking agent comprises an enzyme.

105. The watermarked chemical composition of claim 95, wherein said watermarking agent is an enzyme substrate.

106. The watermarked chemical composition of claim 95, wherein said watermarking agent is an enzyme substrate is configured to be processed to produce said detectable signal.

107. The watermarked chemical composition of claim 95, wherein said watermarking agent comprises one or more components for performing a reaction that produces said detectable signal.

108. The watermarked chemical composition of claim 107, wherein said components comprise nucleic acids and said reaction comprises a nucleic acid extension.

109. The watermarked chemical composition of claim 95, wherein said watermarking agent is configured to produce said detectable signal upon solvation of said watermarking agent.

110. The watermarked chemical composition of claim 95, wherein said detectable signal comprises electromagnetic radiation.

111. The watermarked chemical composition of claim 110, wherein said electromagnetic radiation comprises light.

112. The watermarked chemical composition of claim 95, wherein said detectable signal comprises electromagnetic radiation at a particular wavelength.

113. The watermarked chemical composition of claim 95, wherein said detectable signal comprises electromagnetic radiation at a plurality of wavelengths.

114. The watermarked chemical composition of claim 95, wherein said detectable signal comprises a magnetic field.

115. The watermarked chemical composition of claim 95, wherein said detectable signal comprises an electric field.

116. The watermarked chemical composition of claim 95, wherein said detectable signal comprises a radioactive decay.

117. The watermarked chemical composition of claim 95, wherein said detectable signal comprises a change in signal from a baseline signal.

118. The watermarked chemical composition of claim 117, wherein said change in signal from a baseline signal comprises a decrease in signal amplitude.

119. The watermarked chemical composition of claim 95, further comprising introducing a second watermarking agent.

120. The watermarked chemical composition of claim 119, wherein the combination of said first and second watermarking agents generates said detectable signal upon application of said reaction conditions to the chemical composition.

121. The watermarked chemical composition of claim 119, wherein said second watermarking agent is configured to produce a second detectable signal upon application of reaction conditions to the chemical composition.

122. The watermarked chemical composition of claim 119, wherein said chemical composition comprises said watermarking agent at a first concentration and said second watermarking agent at a second concentration.

123. The watermarked chemical composition of claim 119, wherein said watermarking agent and said second watermarking reagent are configured to produce a detectable signal and a second detectable signal at a ratio of said detectable signal and a second detectable signal; and wherein said ratio indicates that the chemical composition has been watermarked.

124. The watermarked chemical composition of claim 119, wherein said watermarking agent and said second watermarking agent are configured to be detected simultaneously.

125. The watermarked chemical composition of claim 119, wherein said watermarking agent and said second watermarking agent are configured to be detected sequentially.

126. The watermarked chemical composition of claim 119, wherein said watermarking agent and said second watermarking agent are configured to be detected using the same instrument.

127. The watermarked chemical composition of claim 126, wherein said watermarking agent and said second watermarking agent are configured to be detected using the same fluorescent channel.

128. The watermarked chemical composition of claim 95, wherein said chemical composition further comprises assay reagent(s).

129. The watermarked chemical composition of claim 128, wherein said assay reagent(s) comprise reagent(s) for a nucleic acid extension reaction.

130. The watermarked chemical composition of claim 129, wherein said nucleic acid extension reaction is polymerase chain reaction (PCR).

131. The watermarked chemical composition of claim 128, wherein said assay reagent(s) comprise a buffer, a salt, or an enzyme.

132. The watermarked chemical composition of claim 130, wherein said polymerase chain reaction is a quantitative polymerase chain reaction (qPCR).

133. The watermarked chemical composition of claim 128, wherein said assay reagent(s) comprises a DNA polymerase, a reverse transcriptase, a RNA polymerase, or a combination thereof.

134. The watermarked chemical composition of claim 128, wherein said assay reagent(s) comprise a dNTP, a salt, a buffer, or a combination thereof.

135. The watermarked chemical composition of claim 128, wherein said assay reagents(s) comprise an oligonucleotide primer.

136. The watermarked chemical composition of claim 135, wherein said oligonucleotide primer is configured to target a nucleic acid sequence.

137. The watermarked chemical composition of claim 135, wherein said oligonucleotide primer is configured to hybridize to a nucleic acid sequence.

138. The watermarked chemical composition of claim 128, wherein said assay reagent(s) comprise an oligonucleotide probe.

139. The watermarked chemical composition of claim 138, wherein said oligonucleotide probe is a TaqMan® probe.

140. The watermarked chemical composition of claim 138, wherein said oligonucleotide probe is a molecular beacon.

141. The watermarked chemical composition of claim 138, wherein said oligonucleotide probe comprises a label.

142. The watermarked chemical composition of claim 141, wherein said label and said watermarking agent comprise the same molecular structure.

143. The watermarked chemical composition of claim 141, wherein said label and said watermarking agent comprise a different molecular structure.

144. The watermarked chemical composition of claim 141, wherein said label produces said detectable signal.
